(19) United States
(12) Patent Application Publication
(10) Pub. No.: US 2006/0088533 A1
(43) Pub. Date: Apr. 27, 2006

(54) Methods of treating inflammatory bowel disease

(76) Inventors: Charalabos Pothoulakis, Waban, MA (US); J. Thomas LaMont, West Newton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/245,610

(22) Filed: Oct. 7, 2005

Related U.S. Application Data
(60) Provisional application No. 60/617,385, filed on Oct. 8, 2004.

(51) Int. Cl. A61K 39/395; A61K 38/18

(52) U.S. Cl. 424/145.1; 514/2

(57) ABSTRACT

Methods for treating intestinal inflammation by inhibiting Clostridium difficile toxin B-mediated activation of the epidermal growth factor receptor or by inhibiting Clostridium difficile toxin B-mediated activation of the extracellular signal-regulated kinase 1/2 are described.

### RELATED APPLICATION

This application claims the benefit of U.S. Provisional Application No. 60/617,385, filed on Oct. 8, 2004. The entire teachings of the above application are incorporated herein by reference.

### GOVERNMENT SUPPORT

The invention was supported, in whole or in part, by NIH-NIDDK grant R37 DK 34583-21 and NIH-NIDDK grant R01 DK 47343 from National Institutes of Health. The United States Government has certain rights in the invention.

### BACKGROUND OF THE INVENTION

Clostridium difficile, the causative agent of antibiotic-associated colitis in humans, is now recognized as the commonest enteric infections with a 10% symptomatic infection rate for hospitalized patients and an annual cost of over $1.1 billion in the United States (Kyne, L. et al., Clin. Infect. Dis., 34:346-353 (2002)). Toxin A (TxA) and Toxin B (TxB), two high molecular weight exotoxins released from C. difficile, are responsible for the massive fluid secretion, necrosis of the colonic surface mucosa, and acute inflammatory infiltrate (Lamont, J. T., Trans. Am. Clin. Climatol. Assoc., 113:167-180 (2002); Castagliuolo, I. et al., Am. J. Physiol., 273:G333-G341 (1997); Pothoulakis, C. et al., Gastroenterol. Clin. North Am., 22:623-637 (1993); Kelly, C. P. et al., N. Engl. J. Med., 330:257-262 (1994)). These toxins share approximately 63% amino acid homology and have identical glucosyltransferase activity against Rho proteins that leads to disaggregation of the actin cytoskeleton in target cells (von Eichel-Streiber, C. et al., Med. Microbiol. Immunol. (Berl), 179:271-279 (1990)). Both toxins possess cytotoxic activity for cultured mammalian cells, including those derived from the human intestine (Riegler, M. et al., J. Clin. Invest., 95:2004-2011 (1995)). TxA was assumed to be the main factor in causing diarrhea and colitis based on animal studies showing that purified TxA but not TxB caused inflammation and secretion of fluid. The recent isolation of TxA negative/TxB positive strains of C. difficile from patients with antibiotic-associated diarrhea and colitis suggests that TxB alone is sufficient to cause colitis (Kato, H. et al., J. Clin. Microbiol., 36:2178-82 (1998); Limaye, A. P. et al., J. Clin. Microbiol., 38:1696-1697 (2000); Alfa, M. J. et al., J. Clin. Microbiol., 38:2706-2714 (2000)). Moreover, TxB is more potent than TxA in damaging human colonic explants in vivo (Riegler, M. et al., J. Clin. Invest., 95:2004-2011 (1995)).

C. difficile colitis in experimental models and in man requires that the toxins bind their receptors on the luminal aspect of the plasma membrane. Receptor binding is followed by internalization of the toxin, release of reactive oxygen species, activation of nuclear factor κB (NFκB), and subsequent release of interleukin (IL)-8 and other inflammatory cytokines (Castagliuolo, I. et al., Keio J. Med., 48:169-174 (1999)). However, the signaling pathways mediating these responses are largely unknown, particularly for TxB which has no enterotoxic activity in rodent models and rabbits.

### SUMMARY OF THE INVENTION

As described herein, it was discovered that C. difficile TxB signals acute pro-inflammatory responses in colonocytes by transactivation of the epidermal growth factor receptor (EGFR) and activation of the extracellular signal-regulated kinase (ERK)/MAP kinase signaling pathway. TxB induces EGFR phosphorylation in human cultured colonocytes that mediates subsequent activation of ERK1/2 MAP kinase by metalloproteinase (MMP)-mediated transforming growth factor alpha (TGF-α) release. It was also discovered that TxB-mediated EGFR and ERK MAP kinase activation are linked to increased IL-8 gene expression, a major pro-inflammatory cytokine in the pathophysiology of C. difficile-associated colitis.

The present invention provides methods of treating intestinal inflammation (gut inflammation) in a mammal comprising administering to the mammal an effective amount of an agent that inhibits Clostridium difficile toxin B-mediated activation of the epidermal growth factor receptor or Clostridium difficile toxin B-mediated activation of the extracellular signal-regulated kinase 1/2. In one embodiment, intestinal inflammation is a Clostridium difficile toxin B-mediated intestinal inflammation. In another embodiment, intestinal inflammation is an epidermal growth factor receptor-mediated intestinal inflammation. Intestinal inflammation may be associated with, for example, any form of inflammatory diarrhea of the large and/or small bowel, including inflammatory bowel disease (e.g., ulcerative colitis, Crohn's disease), acute enterocolitis, autoimmune inflammation or chronic enterocolitis.

Agents that inhibit Clostridium difficile toxin B-mediated activation of the epidermal growth factor receptor or Clostridium difficile toxin B-mediated activation of the extracellular signal-regulated kinase 1/2 include epidermal growth factor receptor kinase inhibitors, epidermal growth factor receptor antagonists, epidermal growth factor receptor antibodies or antigen-binding fragments thereof, epidermal growth factor receptor inhibitors, extracellular signal-regulated kinase 1/2 inhibitors, extracellular signal-regulated kinase 1/2 antagonists, and matrix metalloproteinase inhibitors. Agents that inhibit Clostridium difficile toxin B-mediated activation of the epidermal growth factor receptor or Clostridium difficile toxin B-mediated activation of the extracellular signal-regulated kinase 1/2 include agents that inhibit binding of TGFα to the epidermal growth factor receptor, such as TGFα antagonists, TGFα antibodies or antigen-binding fragments thereof and TGFα inhibitors. Agents that inhibit Clostridium difficile toxin B-mediated activation of the epidermal growth factor receptor or Clostridium difficile toxin B-mediated activation of the extracellular signal-regulated kinase 1/2 also include agents that inhibit binding of Clostridium difficile toxin B to its cognate cell surface receptor.

Methods are also provided herein for treating Clostridium difficile toxin B-mediated inflammatory diarrhea, including Clostridium difficile toxin B-mediated inflammatory bowel disease (e.g., ulcerative colitis, Crohn's disease), and acute or chronic enterocolitis in a patient by inhibiting Clostridium difficile toxin B-mediated activation of the epidermal growth factor receptor or Clostridium difficile toxin B-mediated activation of the extracellular signal-regulated kinase 1/2. In one embodiment, the methods comprise administering to the patient an effective amount of an epidermal growth factor receptor kinase inhibitor, an epidermal growth factor receptor antagonist (e.g., an epidermal growth factor receptor antibody or antigen-binding fragment thereof, epidermal growth factor receptor inhibitor, small molecule), an extracellular signal-regulated kinase 1/2 inhibitor or an extracellular signal-regulated kinase 1/2 antagonist. In another embodiment, the methods comprise administering to the patient an effective amount of a matrix metalloproteinase inhibitor. In yet another embodiment, the methods comprise administering to the patient an effective amount of an agent that inhibits binding of TGFα to the epidermal growth factor receptor (e.g., a TGFα antagonist, such as a TGFα antibody or antigen-binding fragment thereof, TGFα inhibitor, small molecule). In still another embodiment, the methods comprise administering to the patient an effective amount of an agent that inhibits binding of Clostridium difficile toxin B to its cognate cell surface receptor.

### BRIEF DESCRIPTION OF THE DRAWINGS

FIGS. 1a to 1d depict results showing that TxB induces activation (phosphorylation) of EGFR and ERK. For FIG. 1a, NCM460 cells were incubated with TxB (20 nM) for various times. Cell lysates were resolved on SDS-PAGE gels and immunoblotted against phosphorylated EGFR and ERK1/2. Antibodies against total EGFR or ERK1/2 were used to demonstrate equal protein loading. For FIG. 1b, NCM460 cells were exposed to different concentrations of TxB (1.0 nM-40 nM) for 1 hour. Both phosphorylated and total EGFR and ERK1/2 were detected. For FIG. 1c, HT29 cells were incubated with TxB (20 nM). Activations of EGFR and ERK1/2 were measured as described in FIG. 1a. For FIG. 1d, NCM460 cells were exposed to TxA (20 nM) for different time periods. Activations of EGFR and ERK1/2 levels were determined as described in FIG. 1a.

FIGS. 2a to 2d depict results showing that activation of ERK1/2 is dependent on EGFR phosphorylation. For FIG. 2a, NCM460 cells were pretreated with AG1478 at indicated concentrations for 30 minutes and then exposed to TxB (20 nM) for 60 minutes. Equal amounts of cell lysates were loaded on the SDS-PAGE gels and immunoblotted using antibodies against phosphorylated EGFR and ERK1/2. Total EGFR and ERK1/2 were detected as loading controls. For FIG. 2b, NCM460 cells were preincubated with control IgG (20 μg/ml) or neutralizing antibodies against EGFR (20 μg/ml) for 30 minutes and then exposed to TxB (20 nM) for 1 hour. EGFR and ERK1/2 activation were detected as above. *, P<0.05, cells pretreated with neutralizing antibodies against EGFR versus cells pretreated with control IgG in TxB exposed cells. For FIG. 2c, NCM460 cells were preincubated with PTX (100 ng/ml) overnight and then exposed to TxB (20 nM) for 60 minutes or lysophosphatidic acid (LPA, 25 μM) for 10 minutes. ERK1/2 phosphorylation was detected as above. For FIG. 2d, NCM460 cells were preincubated with medium, neutralizing antibodies against EGFR (20 μg/ml) or AG1478 (0.5 μM) for 30 minutes and then exposed to TxB (20 nM) for 1 hour. Cell extracts were incubated with C. botulinum C3 exoenzyme and 6-Biotin-17-NAD to ADP-ribosylate Rho. Proteins were loaded on the SDS-PAGE gels and immunoblotted using Streptavidin conjugated with Alkaline Phosphatase. Cell rounding was quantitated by phase-contrast microscopy.

FIGS. 3a to 3b depict results showing that EGFR and ERK1/2 activation are prevented by antibodies against TGF-α. NCM460 cells were preincubated with control IgG (20 μg/ml), or neutralizing antibodies against EGF (20 μg/ml), HB-EGF (20 μg/ml), TGF-α (20 μg/ml), or amphiregulin (AR) (20 μg/ml) for 30 minutes and then exposed to TxB (20 nM) for 1 hour. Activations of EGFR (FIG. 3a) and ERK (FIG. 3b) were detected as described in FIGS. 2a to 2d. *, P<0.05, cells treated with neutralizing antibodies against TGF-α versus cells treated with control IgG in TxB exposed groups.

FIGS. 4a to 4b depict results showing that matrix metalloproteinase (MMP) inhibitors prevent EGFR and ERK1/2 activation induced by TxB. NCM460 cells were incubated with either vehicle DMSO, or different MMP inhibitors: batimastat (BB94) (3 mg/ml) or GM6001 (25 μM) for 30 minutes prior to the exposure of TxB (20 nM). Activations of EGFR (FIG. 4a), ERK1/2 (FIG. 4b) were detected by immunoblotting using their phosphorylation specific antibodies. Antibodies against total EGFR and ERK1/2 were used to shown equal amounts of protein were loaded into each lane. *, P<0.05, cells pretreated with BB94 or GM6001 versus cells pretreated with DMSO at 60 minutes in TxB exposed groups.

FIGS. 5a to 5b depict results showing that TxB-mediated TGF-α release is dependent on MMP activation. For FIG. 5a, NCM460 cells were exposed to TxB (20 mM) for various periods of time. TGF-α released into the medium was measured by ELISA. *, P<0.05, TGF-α secretion at indicated time points versus TGF-α secretion at 0 minutes with TxB stimulation. For FIG. 5b, cells were preincubated with DMSO or GM6001 (25 μM) for 30 minutes prior to exposure with TxB for 1 or 3 hours. The concentrations of TGF-α secreted in the conditioned media were determined by ELISA. *, P<0.05, TGF-α secretion with preincubation of GM6001 versus TGF-α secretion with DMSO in TxB exposed groups at indicated time points.

FIGS. 6a to 6b depict results showing that TxB-induced EGFR and ERK activation are required for IL-8 release. For FIG. 6a, NCM460 cells were transiently transfected with IL-8 luciferase promoter along with an internal control. Cells were incubated with TxB (20 nM) at indicated time points. Cells were lysed and luciferase activities were determined. *, P<0.05, IL-8 expression at different time points versus IL-8 expression at 0 minute with TxB exposure. For FIG. 6b, NCM460 cells were transiently transfected with IL-8 luciferase promoter. Cells were preincubated with vehicle DMSO, AG1478 or PD98059 for 30 minutes and exposed with TxB (20 nM) for 16 hours. Cells were lysed and luciferase activities were determined. *, P<0.05, IL-8 expression with pretreatment with AG1478 or PD98059 versus IL-8 expression with pretreatment with DMSO in TxB exposed groups.

FIG. 7 depicts a model of TxB-induced EGFR and ERK activation. Activation of the EGFR and ERK signaling pathway by TxB is shown in the following steps: 1) Stimulation with TxB results in activation of MMPs; 2) MMPs cleave the precursor of TGF-α and release mature TGF-α; 3) Mature TGF-α then binds to the EGFR leading to EGFR phosphorylation; 4) EGFR phosphorylation activates ERK1/2; 5) TxB-mediated EGFR and ERK phosphorylations induce IL-8 expression.

### DETAILED DESCRIPTION OF THE INVENTION

Toxin A (TxA) and Toxin B (TxB), two high molecular weight exotoxins released from C. difficile, are responsible for the massive fluid secretion, necrosis of the colonic surface mucosa, and acute inflammatory infiltrate (Lamont, J. T., Trans. Am. Clin. Climatol. Assoc., 113:167-180 (2002); Castagliuolo, I. et al., Am. J. Physiol., 273:G333-G341 (1997); Pothoulakis, C. et al., Gastroenterol. Clin. North Am., 22:623-637 (1993); Kelly, C. P. et al., N. Engl. J. Med., 330:257-262 (1994)). These toxins share approximately 63% amino acid homology and have identical glucosyltransferase activity against Rho proteins that leads to disaggregation of the actin cytoskeleton in target cells (von Eichel-Streiber, C. et al., Med. Microbiol. Immunol. (Berl), 179:271-279 (1990)). Although receptors for TxA and TxB have not yet been identified, both toxins appear to be widely distributed on enterocytes, fibroblasts, smooth muscle cells and monocytes. Both toxins possess cytotoxic activity for cultured mammalian cells, including those derived from the human intestine (Riegler, M. et al., J. Clin. Invest., 95:2004-2011 (1995)). TxA was assumed to be the main factor in causing diarrhea and colitis based on animal studies showing that purified toxin A but not toxin B caused inflammation and secretion of fluid. The recent isolation of TxA negative/TxB positive strains of C. difficile from patients with antibiotic-associated diarrhea and colitis suggests that TxB alone is sufficient to cause colitis (Kato, H. et al., J. Clin. Microbiol., 36:2178-82 (1998); Limaye, A. P. et al., J. Clin. Microbiol., 38:1696-1697 (2000); Alfa, M. J. et al., J. Clin. Microbiol., 38:2706-2714 (2000)). Moreover, TxB is more potent than TxA in damaging human colonic explants in vivo (Riegler, M. et al., J. Clin. Invest., 95:2004-2011 (1995)). In a xenograft animal model, TxB, like TxA, induced intestinal epithelial cell damage, increased mucosal permeability, stimulated interleukin (IL)-8 synthesis, and elicited an acute inflammatory response (Savidge, T. C. et al., Gastroenterology, 125:413-420 (2003)). However, the precise cellular mechanism(s) and signaling pathway(s) of TxB-mediated inflammation are largely unknown.

The ERK MAP kinase signaling pathway regulates many important cellular processes including proliferation, differentiation and inflammation (Lee, J. C. et al., Nature, 372:739-746 (1994); Han, J. et al., Science, 265:808-811 (1994)). Transactivation of the EGFR has been shown to mediate ERK MAP kinase phosphorylation in response to extracellular stimuli including bacteria, bacterial products, and ligands for G protein-coupled receptors (Daub, H. et al., Nature, 379:557-560 (1996); Pai, R. et al., Nat. Med., 8:289-293 (2002); Castagliuolo, I. et al., J. Biol. Chem., 275:26545-26550 (2000); Keates, S. et al., J. Biol. Chem., 276:48127-48134 (2001)).

The results herein demonstrate that TxB activates ERK MAP kinase in human colonic epithelial cells via a pathway dependent on EGFR tyrosine phosphorylation induced by MMP-mediated TGF-α secretion. The results herein also demonstrate that TxB-mediated EGFR and ERK MAP kinase activation are linked to increased IL-8 gene expression, a major pro-inflammatory cytokine in the pathophysiology of C. difficile-associated colitis.

As described herein, the effect of TxB on EGFR, ERK 1/2 signaling pathway and IL-8 gene expression was assessed in nontransformed human colonic epithelial NCM460 cells. TxB regulation of EGFR-ERK1/2 signaling pathways was determined using immunoblot analysis, confocal microscopy and enzyme-linked immunosorbent assay (ELISA). IL-8 gene expression was measured by luciferase promoter assay.

The results herein show that TxB activates the EGFR and ERK1/2 phosphorylation with subsequent release of IL-8 from human colonocytes. Pretreatment with either the EGFR tyrosine kinase inhibitor, AG1478 or an EGFR neutralizing antibody blocked both TxB-induced EGFR and ERK activation. Using neutralizing antibodies against known ligands of EGFR, it was found that the activation of EGFR and ERK1/2 phosphorylation was mediated by TGF-α. Inhibition of MMP decreased TGF-α secretion and TxB-induced EGFR and ERK activation. Inhibition of EGFR and ERK phosphorylation significantly decreased TxB induced IL-8 expression. These results show that TxB signals acute pro-inflammatory responses in colonocytes by transactivation of the EGF receptor and activation of the ERK/MAP kinase pathway.

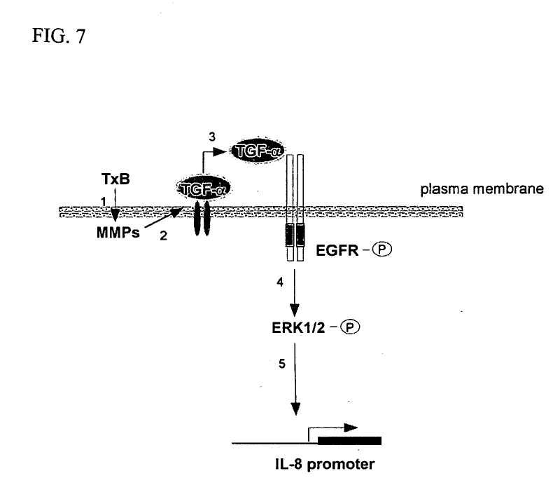

The present invention relates to a novel signaling pathway for TxB of C. difficile in human colonic epithelial cells (FIG. 7), the natural target cell of this bacterial toxin in human disease.

The present invention provides methods of treating intestinal inflammation (gut inflammation) in a mammal comprising administering to the mammal an effective amount of an agent that inhibits Clostridium difficile toxin B-mediated activation (phosphorylation) of the epidermal growth factor receptor (EGFR) or Clostridium difficile toxin B-mediated activation (phosphorylation) of the extracellular signal-regulated kinase (ERK) 1/2. In one embodiment, intestinal inflammation is a Clostridium difficile toxin B-mediated intestinal inflammation. In another embodiment, intestinal inflammation is an epidermal growth factor receptor-mediated intestinal inflammation. Intestinal inflammation may be associated with, for example, any form of inflammatory diarrhea of the large and/or small bowel, including inflammatory bowel disease (e.g., ulcerative colitis, Crohn's disease), acute enterocolitis, autoimmune inflammation or chronic enterocolitis.

Agents that inhibit Clostridium difficile toxin B-mediated activation of the EGFR or Clostridium difficile toxin B-mediated activation of the ERK 1/2 include EGFR kinase inhibitors (such as tyrphostin AG1478), EGFR antagonists, EGFR antibodies or antigen-binding fragments thereof, EGFR inhibitors, ERK 1/2 inhibitors (such as PD98059), ERK 1/2 antagonists, and matrix metalloproteinase (MMP) inhibitors (such as GM6001). Agents that inhibit Clostridium difficile toxin B-mediated activation of the EGFR or Clostridium difficile toxin B-mediated activation of the ERK 1/2 include agents that inhibit binding of TGFα to the EGFR, such as TGFα antagonists, TGFα antibodies or antigen-binding fragments thereof, and TGFα inhibitors. Agents that inhibit Clostridium difficile toxin B-mediated activation of the EGFR or Clostridium difficile toxin B-mediated activation of the ERK 1/2 also include agents that inhibit binding of Clostridium difficile toxin B to its cognate cell surface receptor.

Methods are also provided herein for treating Clostridium difficile toxin B-mediated inflammatory diarrhea, including Clostridium difficile toxin B-mediated inflammatory bowel disease (e.g., ulcerative colitis, Crohn's disease), and acute or chronic enterocolitis in a patient by inhibiting Clostridium difficile toxin B-mediated activation of the epidermal growth factor receptor or Clostridium difficile toxin B-mediated activation of the extracellular signal-regulated kinase 1/2. In one embodiment, the methods comprise administering to the patient an effective amount of an epidermal growth factor receptor kinase inhibitor, an epidermal growth factor receptor antagonist (e.g., an epidermal growth factor receptor antibody or antigen-binding fragment thereof, epidermal growth factor receptor inhibitor, small molecule), an extracellular signal-regulated kinase 1/2 inhibitor or an extracellular signal-regulated kinase 1/2 antagonist. In another embodiment, the methods comprise administering to the patient an effective amount of a matrix metalloproteinase inhibitor. In yet another embodiment, the methods comprise administering to the patient an effective amount of an agent that inhibits binding of TGFα to the epidermal growth factor receptor (e.g., a TGFα antagonist, such as a TGFα antibody or antigen-binding fragment thereof, TGFα inhibitor, small molecule). In still another embodiment, the methods comprise administering to the patient an effective amount of an agent that inhibits binding of Clostridium difficile toxin B to its cognate cell surface receptor.

The term “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and primates (e.g., monkeys, chimpanzees), rodents (e.g., rats, mice, guinea pigs) and ruminents (e.g., cows, pigs, horses). The term “patient”, as used herein, refers to a mammalian subject in need of treatment, including human subjects.

An agent “inhibits” activity if it blocks, interferes with, decreases or abrogates the activity which would be exhibited in the absence of the agent. For example, inhibitors decrease or block activity, e.g., functional inhibitors that interact and block an active site, or competitive inhibitors that compete for binding; and antagonists inhibit binding activity, e.g., molecules that reduce binding affinity between a receptor and ligand. Examples of such molecules include, but are not limited to, antibodies, small molecule agents, antagonists, non-biologically active analogs, receptor antagonists. These agents can be proteins, peptides, peptide analogs, or chemical compounds or derivatives.

Agents can be administered alone (naked administration) or as part of a composition. Routes of administration are generally known in the art and include aerosol, oral, systematic, intravenous including infusion and/or bolus injection, intrathecal, parenteral, mucosal, implant, intraperitoneal, intradermal, transdermal (e.g., in slow release polymers), intramuscular, subcutaneous, topical, epidural, etc. routes. Other suitable routes of administration can also be used, for example, to achieve absorption through epithelial or mucocutaneous linings. Agents of the present invention can also be administered by gene therapy, wherein a DNA molecule encoding a particular therapeutic protein or peptide is administered to the patient, e.g., via a vector, which causes the particular protein or peptide to be expressed and secreted at therapeutic levels in vivo.

Agents can be administered together with other components of biologically active agents, such as pharmaceutically acceptable surfactants (e.g., glycerides), excipients (e.g., lactose), carriers, diluents and vehicles. If desired, certain sweetening, flavoring and/or coloring agents can also be added.

Agents can be administered prophylactically or therapeutically to a patient prior to, simultaneously with or sequentially with other therapeutic regimens or agents (e.g., multiple drug regimens), including with other therapeutic regimens used for the treatment of inflammation, including gut inflammation and related disorders. Agents that are administered simultaneously with other therapeutic agents can be administered in the same or different compositions.

It may be undesirable to administer the protein systemically because of side-effects. To eliminate pleiotropic effects of administering an agent in accordance with the present invention, it would be useful to deliver (or target) the protein to a specific tissue (e.g., intestinal tissue, epithelial cells, colonocytes, lamina propria cells). One way to deliver the protein to a specific tissue is to conjugate the protein with a targeting agent. For example, the protein can comprise a peptide to target the cognate receptor to a specific tissue or cell type, e.g., intestinal tissue or cells. Such targeting molecules are well known to those of skill in the art.

Agents can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation can be sterilized by commonly used techniques. Suitable pharmaceutical carriers are described in Remington's Pharmaceutical Sciences.

The term “pharmaceutically acceptable” can be used interchangeably with “physiologically acceptable” to mean a non-toxic material that does not interfere with the effectiveness of the biological activity of the active ingredient(s).

An “effective amount” of agent or composition is defined herein as that amount, or dose, of agent or composition of the invention that, when administered to the subject, is sufficient to reduce or inhibit intestinal inflammation in a specific tissue or cell. The dosage administered to a subject will vary depending upon a variety of factors, including the pharmacodynamic characteristics of the particular agent or composition, and its mode and route of administration; size, age, sex, health, body weight and diet of the recipient; nature and extent of symptoms of the disease or condition being treated, kind of concurrent treatment, frequency of treatment, and the effect desired.

An effective amount can be administered in single or divided doses (e.g., a series of doses separated by intervals of days, weeks or months), or in a sustained release form, depending upon factors such as nature and extent of symptoms, kind of concurrent treatment and the effect desired. Other therapeutic regimens or agents can be used in conjunction the present invention. Adjustment and manipulation of established dosage ranges are well within the ability of those skilled in the art.

Once an effective amount has been administered, a maintenance amount of an agent or composition of the invention can be administered to the subject. A maintenance amount is the amount of agent or necessary to maintain the reduction or inhibition of the inflammatory response mediated by ghrelin and ghrelin receptor in a specific tissue or cell that was achieved by the effective dose. The maintenance amount can be administered in the form of a single dose, or a series of doses separated by intervals of days or weeks (divided doses).

Second or subsequent administrations can be administered at a dosage which is the same, less than or greater than the initial or previous dose administered to the subject. A second or subsequent administration is preferably during or immediately prior to relapse or a flare-up of the condition. For example, the second and subsequent administrations can be given between about one day to 30 weeks from the previous administration. Two, three, four or more total administrations can be delivered to the subject, as needed.

Dosage forms (compositions) suitable for internal administration generally contain from about 0.1 milligram to about 500 milligrams of active ingredient per unit. In these pharmaceutical compositions the active ingredient will ordinarily be present in an amount of about 0.5-95% by weight based on the total weight of the composition.

The present invention will now be illustrated by the following examples, which are not to be considered limiting in any way.

### EXAMPLES

Material And Methods

Toxin A (TxA) And Toxin B (TxB) Preparation And Cell Culture

TxA and TxB were purified from C. difficile strain 10463 (ATCC, Manassas, Va.) as described previously (Chen, M. L. et al., J. Biol. Chem., 277:4247-4254 (2002); He, D. et al., Gastroenterology, 119:139-50 (2000); Warny, M. et al., J. Clin. Invest., 105:1147-56 (2000)). Purified toxins A or B were suspended in Tris-HCL (PH=7.5) and piperazine-HCL (PH=5.5), respectively, at a concentration of 10 μM. Nontransformed human colonic epithelial cells NCM460 were maintained in M3D medium (INCELL Corporation, San Antonio, Tex.). HT29 cells derived from human colorectal adenocarcinoma were maintained in McCoy's 5A medium (Invitrogen, Carlsdad, Calif.). Cells were cultured in a 37° C. humidified incubator with 5% CO₂.

Antibodies And Reagents

Total and phosphorylated EGFR antibodies were from Santa Cruz Biotechnology (Santa Cruz, Calif.). Antibodies against ERK1/2 and phospho-specific p44/p42 MAP kinase were from Cell Signaling Technology (Beverly, Mass.). Heparin-binding-EGF (HB-EGF), TGF-α, Amphiregulin, and their respective neutralizing antibodies were from R&D Systems (Minneapolis, Minn.). EGF and EGFR neutralizing antibodies were from Sigma (St. Louis, Mo.) and Upstate Biotechnology (Charlottesville, Va.), respectively. AG1478, BAPTA, GF109203X, GM6001, and inactive GM6001 control compound were from Calbiochem (La Jolla, Calif.) and batimastat (BB94) was from British Biotech (Oxford, UK).

Western Blot Analysis

NCM460 or HT29 cells were seeded in 12 well plates and grown to 70%-100% confluency. Cells were serum starved overnight prior to experiments. TxB or TxA were added in serum free medium at the indicated concentrations for varying lengths of time. Cells were then washed three times with ice cold phosphate buffered saline (PBS) and lysed in 1×SDS sample buffer (Cell Signaling Technology, Beverly, Mass.). Equal amounts of cell lysates were loaded onto 10% SDS-PAGE gels and transferred to nitrocellulose membranes (Bio-Rad Laboratories, Hercules, Calif.). Blots were probed with each primary antibody overnight at 4° C. and then with species-specific secondary antibodies coupled with horseradish peroxidase for 1 hour at room temperature. Peroxidase activity was detected by SuperSignal Chemiluminescent Substrate (Pierce, Rockford, Ill.). Signals were analyzed and quantified as described (Na, X. et al., EMBO J., 22:4249-4259 (2003)) and results were expressed as mean ±SD.

C3 Exoenzyme-Catalyzed ADP-Ribosylation Assay

The C3 exo enzyme of Clostridium botulinum (C. botulinum) ADP-ribosylates Rho at Asn41, a reaction that is inhibited by prior monoglucosylation of Rho by TxB (Just, I. et al., J. Biol. Chem., 270:13932-13936 (1995); Just, I. et al., Nature, 375:500-503 (1995); Hofmann, F. et al., J. Biol. Chem., 272:11074-11078 (1997)). Thus, inhibition of C3 is used to estimate TxB inactivation of Rho. NCM460 cells were seeded on 100 mm plates and grown to 90%-100% confluency. Cells were incubated with EGFR antibody or AG1478 for 30 minutes in the indicated groups and then exposed to TxB for 60 minutes. Cells were resuspended in 100 ul Lysis buffer (Warny, M. et al., J. Clin. Invest., 105:1147-1156 (2000)), frozen and thawed twice and centrifuged at 14,000 rpm for 10 minutes to remove insoluble materials. The C. botulinum C3-catalyzed ADP-ribosylation of Rho was initiated by adding 37 μl extract to 10 μl of 5× C3 buffer (Warny, M. et al., J. Clin. Invest., 105:1147-1156 (2000)), 10 μM 6-Biotin-17-AND (Zhang J., Methods Enzymol., 280:255-265 (1997) (Trevigen, Gaithersburg, Md.), and 50 ng C3 (Biomol Research Laboratories, Plymouth Meeting, Pa.) in a final volume of 50 μl. After incubation for 1 hour at 37° C., the reaction was stopped by adding 25 μl 3×SDS sample buffer. Extracts were subjected to 12% SDS-PAGE, and ADP-ribosylated Rho was immunobloted by 1:1000 Streptavidin conjugated with Alkaline Phosphatase (Pierce Biotechnology, Rockford, Ill.).

Cell Rounding Assay

NCM460 cells were seeded on glass cover slides overnight. Cells were incubated with EGFR antibody or AG1478 for 30 minutes and exposed to TxB for 1 hour. Cells were then fixed with formaldehyde for 10 minutes and rinsed three times in PBS. Percentage of cell rounding was quantitated by phase-contrast microscopy.

Enzyme-Linked Immunosorbent Assay (ELISA)

TGF-α secretion from colonocyte cultures was measured by enzyme-linked immunosorbent assay. Briefly, 96-well Nunc-immuno plates (Fisher Scientific) were coated with 0.4 ug/ml of TGF-α antibody diluted in PBS overnight at room temperature. Wells were blocked with PBS containing 1% BSA for 1 hour after washing 3 times with PBST (PBS with 0.1% Twin-20). Fifty μl of conditioned media or serial dilutions of TGF-α in PBS containing 1% BSA were then added to each well. After 2 hours, wells were washed and incubated with 0.3 μg/ml biotin-conjugated anti-TGF-α antibody for 1 hour. Streptavidin-horseradish peroxidase (1:1500) from Amersham Bioscience (Amersham, UK) was added to the wells. Peroxidase activity was measured by TMB Peroxidase Substrate Reagents (KPL Inc, Gaithersburg, Md.).

Luciferase Promoter Assay

A 1521-bp fragment (nucleotides −1481 to +40) of the promoter region of the human IL-8 gene was used to monitor transcription of IL-8 gene as previously described (Keates, S. et al., J. Biol. Chem., 276:48127-48134 (2001)). Cells were grown on 24-well plates and transiently transfected with the IL-8 promoter luciferase construct and a control luciferase construct pRL-TK (Promega Corp., Madison, Wis.) using Superfect (Qiagen Inc., Chatsworth, Calif.). Transfected cells were serum starved overnight and then exposed to TxB and/or pharmacological inhibitors at the indicated times. Cells were lysed, and firefly and renilla luciferase activities in cell extracts were measured using Dual-Luciferase Reporter Assay System (Promega Corp., Madison, Wis.). The relative luciferase activity was calculated by normalizing IL-8 promoter-driven firefly luciferase activity to control Renilla luciferase activity. Data from all experiments are represented as the relative luciferase activity (mean ±SD) from at least three independent sets of experiments, each with triplicate measurements.

Statistical Analyses

Data were analyzed using the SIGMA-STAT professional statistics software program (Jandel Scientific Software, San Rafael, Calif.). Analyses of variance with protected t test (ANOVA) were used for intergroup comparison.

Results

TxB-Induced Phosphorylation of EGFR And ERK

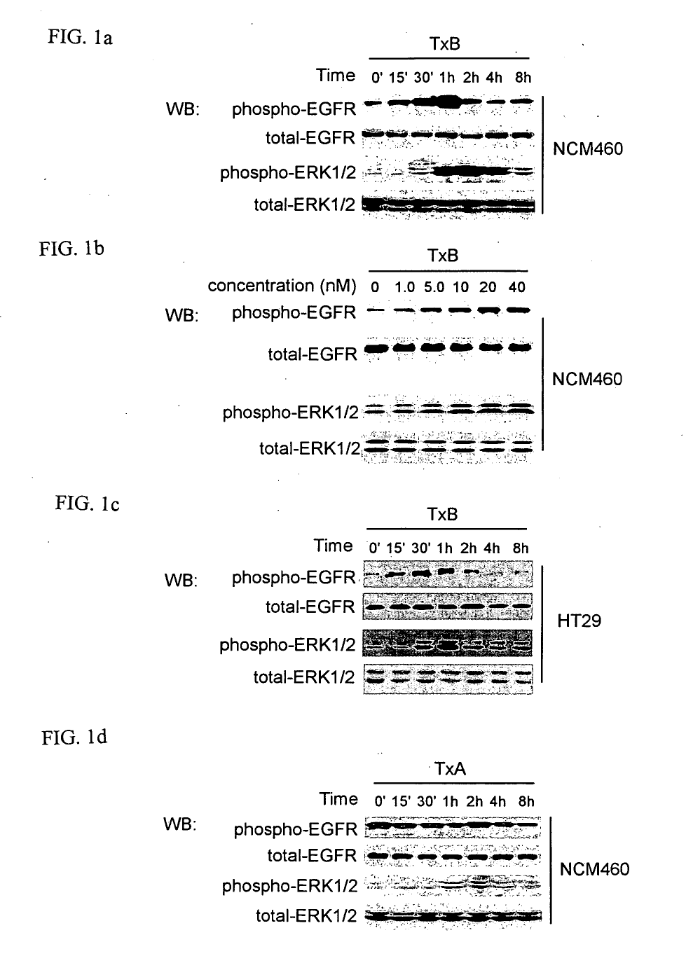

To determine if TxB activates EGFR and ERK, nontransformed human colonocytes were exposed to TxB (20 nM) and EGFR phosphorylation was determined by immunoblotting using antibodies against phosphorylated EGFR. EGF receptor phosphorylation was detected at 15-30 minutes after TxB exposure, and was maximal at 1 hour (FIG. 1a). ERK1/2 phosphorylation levels increased after 30 minutes of TxB exposure and remained elevated for 8 hours (FIG. 1a). The effects of TxB on EGFR and ERK activation also were examined in cells grown on collagen-coated permeable supports. It was found that TxB applied to either the apical or basolateral compartment-activated EGFR and ERK, with a stronger response observed after basolateral vs. apical exposure. Phosphorylation of EGFR and ERK1/2 increased dose-dependently after TxB stimulation with a maximum 20 nM TxB exposure (FIG. 1b). Similar activation of EGFR and ERK by TxB was observed in HT29 colonic adenocarcinoma cells (FIG. 1c).

Although both TxA and TxB inactivate Rho proteins in cells and cause similar effects on cultured cells and human colonic mucosa (Just, I. et al., J. Biol. Chem., 270:13932-13936 (1995); Just, I. et al., Nature, 375:500-503 (1995); Hofmann, F. et al., J. Biol. Chem., 272:11074-11078 (1997)), it is not known if they bind to the same cell surface receptor. To determine whether TxA also induces EGFR and ERK1/2 activation, NCM 460 cells were exposed to TxA for different periods of time and measured phosphorylation of EGFR and ERK1/2. As shown in FIG. 1d, TxA had no observable stimulatory effect on EGFR phosphorylation. However, consistent with our prior observations in human monocytes (Warny, M. et al., J. Clin. Invest., 105:1147-1156 (2000)), ERK1/2 phosphorylation was increased after 1-hour exposure with TxA and declined to basal levels 8 hours after stimulation. These results suggest that TxA and TxB use different receptors and signaling pathways to activate ERK in colonic epithelial cells.

Activation of ERK1/2 is Dependent on EGFR Phosphorylation

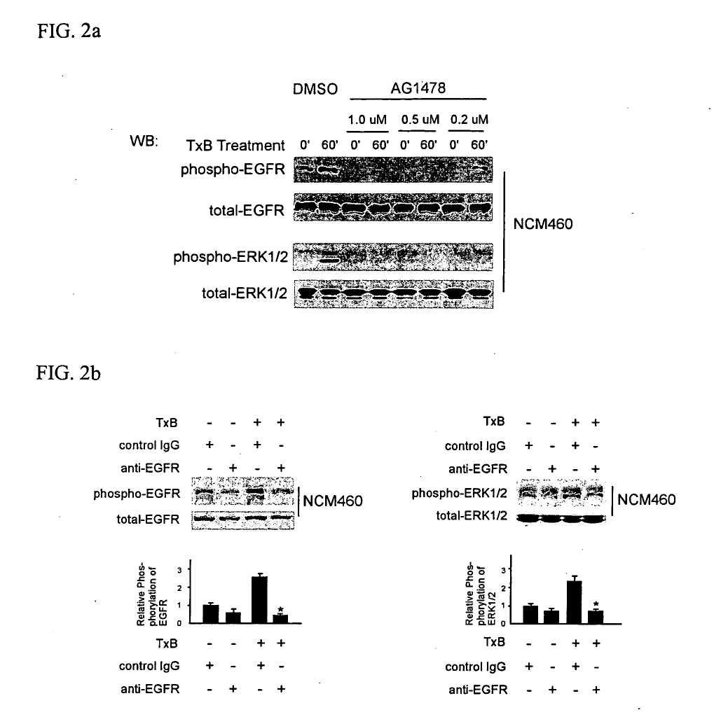

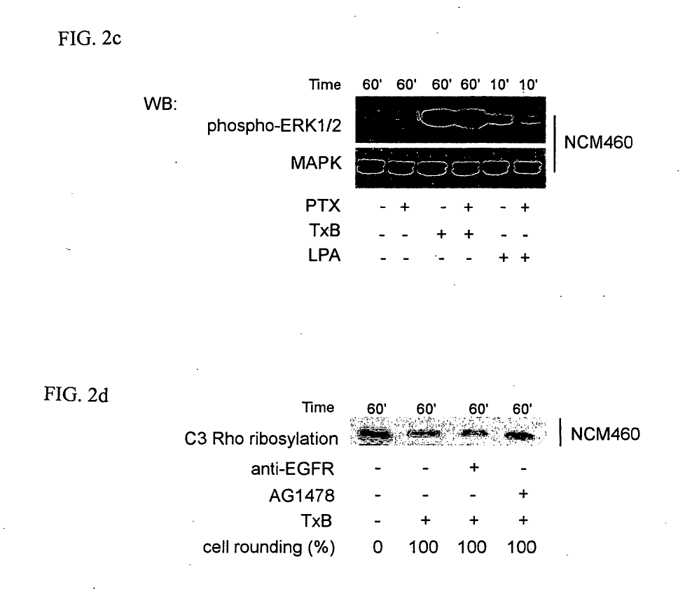

To ascertain the role of TxB-mediated EGFR activation in triggering the ERK1/2 pathway, colonocytes were preincubated with the specific EGFR kinase inhibitor, tyrphostin AG1478, and then examined TxB-mediated ERK1/2 activation. Activation of EGFR and ERK1/2 by TxB was completely blocked after incubation with AG1478 at concentrations of 0.2-1.0 uM (FIG. 2a).

Next it was tested whether TxB-induced activation of EGFR involves direct binding of its ligand, using a specific EGFR antibody that binds the extracellular domain of EGFR and blocks ligand binding. As shown in FIG. 2b, this blocking antibody significantly inhibited TxB-mediated phosphorylation of EGFR and ERK1/2, indicating that ligand binding to EGFR is required for ERK activation in response to TxB.

Numerous G-protein-coupled receptors (GPCR) are known to transactivate EGFR (Luttrell, L. M. et al., Curr. Opin. Cell Biol., 11:177-183 (1999)); Hackel, P. O. et al., Curr. Opin. Cell. Biol., 11:184-189 (1999)). Pertussis toxin (PTX) selectively blocks the G_{i/o} subfamily of G proteins. To test whether G_{i/o} G proteins are involved in TxB-mediated ERK1/2 activation, colonocytes were preincubated with PTX (100ng/ml) overnight and then exposed to TxB (20 nM) or lysophosphatidic acid (LPA, 25 uM), which mediates ERK1/2 activation primarily through PTX-sensitive G_{i/o} proteins (Luttrell, L. M. et al., J. Biol. Chem., 270:16495-16498 (1995); Luttrell, L. M. et al., J. Biol. Chem., 271:19443-19450 (1996)). As shown in FIG. 2c, PTX blocked LPA-mediated ERK1/2 activation but had no effect on TxB-mediated ERK1/2 activation in NCM460 cells.

To test whether TxB-mediated transactivation of the EGFR and ERK involves these mediators, colonocytes were preincubated with BAPTA (20uM), a calcium chelator, and GF109203X (1uM), an inhibitor of PKC, before TxB addition. Both BAPTA and GF109203X decreased TxB's activation of EGFR and ERK.

C. difficile toxins induce early (<30 min) cellular responses prior to Rho inactivation including activation of mitogen-activated protein kinases (Warny, M. et al., J. Clin. Invest., 105:1147-1156 (2000)), generation of reactive oxygen metabolites (He, D. et al., Gastroenterology, 119:139-150 (2000)), induction of calcium influx (Gilbert, R. J. et al., Am. J. Physiol., 268:G487-G495 (1995)), and stimulation the activities of membrane and cytosolic protein kinase Cα (PKCα) and cytosolic PKCβ (Chen, M. L. et al., J. Biol. Chem., 277:4247-4254 (2002)). These findings suggest that Rho-independent early signaling events related to toxin-receptor binding may be involved in pathophysiology of diarrhea and inflammation. To investigate whether TxB-mediated EGFR phosphorylation is required for Rho glucosylation, it was examined whether TxB-induced Rho glucosylation measured by inhibition of C3 toxin-mediated Rho ribosylation and cell rounding could be altered by preventing EGFR activation. As shown in FIG. 2d, neither an EGFR antibody, nor a specific EGFR inhibitor, AG1478, altered TxB-induced Rho glucosylation and cell rounding.

EGFR And ERK1/2 Activation Are Prevented By Antibodies Against TGF-α

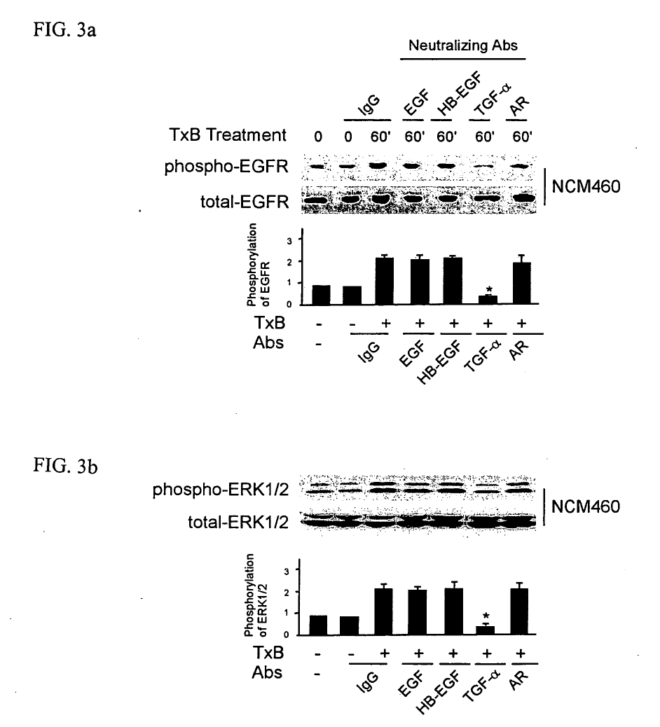

EGFR can be activated by four different ligands: EGF, HB-EGF, TGFα and amphiregulin (AR). To identify which of these ligands mediate TxB-related signaling, colonocytes were treated with neutralizing antibodies against EGF (20 μg/ml), HB-EGF (20 μg/ml), TGF-α (20 μg/ml), AR (20 μg/ml), or their respective control antibodies (IgG) at the same concentration, prior to TxB exposure. Only the TGF-α neutralizing antibody significantly inhibited TxB-induced EGFR (FIG. 3a) and ERK1/2 (FIG. 3b) phosphorylation, indicating that TGF-α mediates TxB-induced EGFR and ERK phosphorylation.

Matrix Metalloproteinase (MMP) Inhibitors Prevent EGFR and ERK1/2 Activation Induced by TxB.

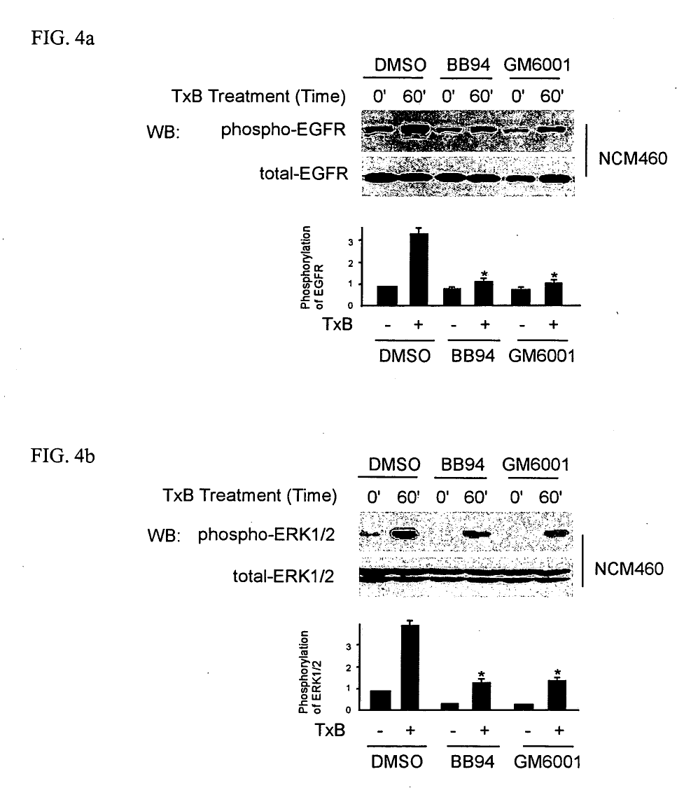

Previous studies have demonstrated that activation of MMPs and subsequent cleavage of EGF ligands are involved in transactivation of EGFR by prostaglandin E2 (Pai, R. et al., Nat. Med., 8:289-293 (2002)), thrombin and endothelin-1 (Luttrell, L. M. et al., Curr. Opin. Cell Biol., 11:177-183 (1999); Hackel, P. O. et al., Curr. Opin. Cell Biol., 11:184-189 (1999); Prenzel, N. et al., Nature, 402:884-888 (1999)), and Helicobacter pylori (Keates, S. et al., J. Biol. Chem., 276:48127-48134 (2001)). To assess the possible involvement of MMPs in TxB-mediated EGFR and ERK activation, NCM460 cells were treated with the MMP inhibitors GM6001 or BB94 prior to TxB stimulation. Activation of the EGFR (FIG. 4a) and ERK1/2 (FIG. 4b) signaling cascade were significantly inhibited after pretreatment with either MMP inhibitor.

TxB-Mediated TGF-α Release is Dependent on MMP Activity

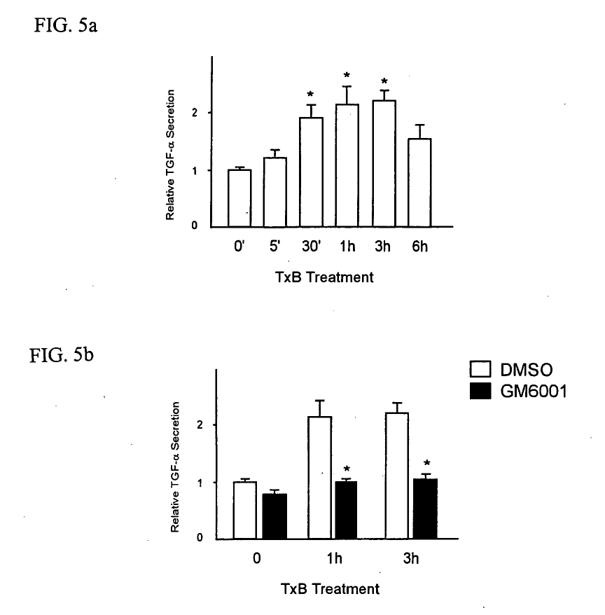

Since both MMP and TGF-α were required for TxB-induced activation of the EGFR and ERK signaling cascades, the relationship between these two factors were examined. First, whether TxB could directly induce TGF-α release was tested. Increased secretion of TGF-α was detected 5 minutes after TxB exposure and gradually reached to its peak at approximately 1 to 3 hours (FIG. 5a), in agreement with the time course of TxB-mediated EGFR and ERK1/2 activation (FIG. 1a). To further test the effect of MMP on the release of TGF-α, GM6001, a MMP inhibitor, was added to cells 30 minutes prior to TxB treatment. As shown in FIG. 5b, GM6001 significantly inhibited TxB-induced TGF-α secretion.

TxB-Induced EGFR And ERK Activation Are Required For IL-8 Release

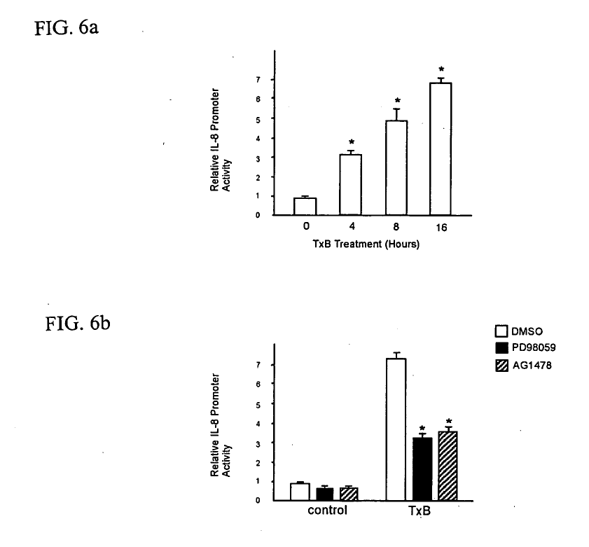

TxB stimulates interleukin IL-8 synthesis, and causes an acute inflammatory response in a human intestinal mouse xenograft model (Savidge, T. C. et al., Gastroenterology, 125:413-420 (2003)). To evaluate the physiological significance of EGFR and ERK activation in response to TxB, it was examined whether they are involved in TxB-induced IL-8 gene expression. Since IL-8 production is regulated primarily at the transcriptional level, the effect of TxB on IL-8 promoter activity in NCM460 cells was tested. As shown in FIG. 6a, TxB increased IL-8 transcription in a time-dependent manner. Next, whether EGFR and ERK1/2 blockade inhibits TxB-mediated IL-8 gene expression was tested. As shown in FIG. 6b, pretreatment of NCM460 cells with the EGFR inhibitor AG1478 or the ERK1/2 inhibitor PD98059 for 30 minutes significantly inhibited TxB-mediated IL-8 expression. Because IL-8 gene expression is often linked to NFκB pathway, TxB-mediated NFκB signaling response in NCM460 cells was tested. No IκB phosphorylation, degradation and NFκB p65 phosphorylation were observed in our study.

Discussion

C. difficile colitis in experimental models and in man requires that the toxins bind their receptors on the luminal aspect of the plasma membrane. Receptor binding is followed by internalization of the toxin, release of reactive oxygen species, activation of nuclear factor κB (NFκB), and subsequent release of IL-8 and other inflammatory cytokines (Castagliuolo, I. et al., Keio J. Med., 48:169-174 (1999)). However, the signaling pathways mediating these responses are largely unknown, particularly for TxB which has no enterotoxic activity in rodent models and rabbits. The results herein demonstrate that C. difficile TxB induces EGFR phosphorylation in human cultured colonocytes that mediates subsequent activation of ERK1/2 MAP kinase by MMP-mediated TGF-α release. The results herein also demonstrate that the EGFR and ERK activation but not NFκB are important for TxB-mediated IL-8 gene expression (FIG. 7).

Differential phosphorylation of EGFR after treatment with TxA and TxB was observed, even though these toxins share close structural and sequence homology by sequence analysis and both possess potent glucosyltransferase activity for Rho proteins (von Eichel-Streiber, C. et al., Med. Microbiol. Immunol. (Berl), 179:271-279 (1990)). TxA does not have detectable effect on EGFR phosphorylation at the same concentration as TxB. TxA and TxB share 63% sequence homology and have similar primary structures and functional domains (von Eichel-Streiber, C. et al., Med. Microbiol. Immunol. (Berl), 179:271-279 (1990)). The enzymatic domain of TxA and B resides in the NH₂-terminal portion, which inactivates Rho GTPases by hydrolyzing UDP-glucose and transferring a glucose moiety to threonine 37 of the small GTP-binding proteins, Rho, Cdc42 and Rac. These covalent modifications of Rho family proteins subsequently induce disaggregation of actin microfilaments and cause cell rounding (Just, I. et al., J. Biol. Chem., 270:13932-13936 (1995); Just, I. et al., Nature, 375:500-503 (1995); Hofmann, F. et al., J. Biol. Chem., 272:11074-11078 (1997)). Although both toxins are able to phosphorylate ERK1/2 in colonic epithelial cells, only TxB activated EGFR, suggesting that these toxins probably bind to separate cell surface receptors and activate different signaling cascades to activate the MAP kinase pathway.

Previous studies have shown that other bacteria or bacteria toxins are able to transactivate EGFR. For example, H. pylori induces the phosphorylation EGFR via activation of the endogenous ligand heparin-binding EGF-like growth factor (Keates, S. et al., J. Biol. Chem., 276:48127-48134 (2001)); Pasteurella multocida toxin stimulates the MAPK pathway via G_{q/11}-dependent transactivation of the EGFR (Seo, B. et al., J. Biol. Chem., 275:2239-2245 (2000)). It is demonstrated here that TxB-mediated EGFR and MAP kinase activation involves induction of MMP-dependent TGF-α release. Once activated, MMPs are able to cleave the membrane-bound precursor of EGFR ligands, and release of their soluble ligands, like TGF-α (Luttrell, L. M. et al., Curr. Opin. Cell Biol., 11:177-183 (1999); Hackel, P. O. et al., Curr. Opin. Cell Biol., 11:184-189 (1999); Prenzel, N. et al., Nature, 402:884-888 (1999)). These in turn activate the EGFR, phosphorylate and activate the MAP kinase cascade.

In colonocytes exposed to TxB, TGF-α release can be detected within 5 minutes (FIG. 5a). Rho glucosylation is typically detected at 30-60 minutes since this process requests toxin internalization and translocation into the cytoplasm (Chen, M. L. et al., J. Biol. Chem., 277:4247-4254 (2002); He, D. et al., Gastroenterology, 119:139-150 (2000); Warny, M. et al., J. Clin. Invest., 105:1147-1156 (2000)). Rapid release of TGF-α suggests that TxB-induced EGFR transactivation precedes Rho glucosylation. Using neutralizing antibody against EGFR and EGFR inhibitor (AG1478), it is demonstrated that inhibition of EGFR activation did not prevent TxB-induced Rho glucosylation but did inhibit IL-8 release (FIG. 6). These findings suggest that initial MAP kinase activation and release of IL-8 are independent of Rho glucosylation.

Transactivation of EGFR by numerous G-protein-coupled receptors (GPCR) has been identified as an important element in GPCR-induced signaling (Luttrell, L. M. et al., Curr. Opin. Cell Biol., 11:177-183 (1999); Hackel, P. O. et al., Curr. Opin. Cell Biol., 11:184-189 (1999)). GPCRs have seven-transmembrane domains and incorporate heterotrimeric G-proteins to generate signals upon activation by a variety of ligands (Luttrell, L. M. et al., Curr. Opin. Cell Biol., 11: 177-183 (1999)). G proteins including G_{s (}Bertelsen, L. S. et al., J. Biol. Chem., 279:6271-6279 (2004)), Gᵢ (Daub, H. et al., Nature, 379:557-560 (1996); Daub, H. et al., EMBO J., 16:7032-7044 (1997)), G_{q/11} (Eguchi, S. et al., J. Biol. Chem., 273:8890-8896 (1998); Roelle, S. et al., J. Biol. Chem., 278:47307-47318 (2003)) and G₁₃ (Gohla, A. et al., J. Biol. Chem., 273:4653-4659 (1998)) have been shown to mediate EGFR transactivation. TxA-stimulated intracellular calcium release from human granulocytes was blocked by pertussis toxin, suggesting that the toxin was binding to a Gᵢ-coupled receptor (Pothoulakis, C. et al., J. Clin. Invest., 81:1741-1745 (1998)). Preincubation of colonocytes with pertussis toxin to selectively block G_{i/o} had no effects on TxB-mediated ERK activation, indicating that the G_{i/o} subfamily of G protein is not involved in the TxB response in NCM460 cells. TxB presumably exerts its function through interacting with a cognate (TxB) receptor expressed on the apical (luminal) surface of intestinal epithelial cells. Whether this TxB receptor is also a GPCR can be determined.

IL-8 is a potent inflammatory cytokine that plays a critical role in TxB-mediated colitis by stimulating neutrophil migration into the diseased segment of bowel. In human intestinal explants xenografted into SCID mice, TxB was found to induce intestinal epithelial cell-specific IL-8 gene expression (Savidge, T. C. et al., Gastroenterology, 125:413-420 (2003)). EGFR transactivation and MAP kinases promote cell proliferation and tissue repair. Recent studies have shown that this pathway also leads to expression of pro-inflammatory cytokines including IL-8 (Keates, S. et al., J. Biol. Chem., 276:48127-48134 (2001)). AG1478, an EGFR phosphorylation inhibitor, significantly reduced IL-8 production mediated by H. pylori (Keates, S. et al., J. Biol. Chem., 276:48127-48134 (2001); Keates, S. et al., J. Immunol., 163:5552-5559 (1999)) and neurotensin (Zhao, D. et al., J. Biol. Chem., Manuscript M401453200, published Jul. 6, 2004). Consistent with these reports, our results indicate that EGFR-ERK signaling pathway is also critical in TxB-mediated IL-8 production. NFκB plays an important role in regulating IL-8 gene expression (Oliveira, I. C. et al., Mol. Cell. Biol., 14:5300-5308 (1994); Kunsch, C. et al., Mol. Cell. Biol., 13:6137-6146 (1993)). NFκB activation is involved C. difficile TxA mediated IL-8 release in enterocytes (He, D. et al., Gastroenterology, 119:139-150 (2000)). However, TxB exposure does not activate NFκB pathway in nontransformed human colonic epithelial cells.

In conclusion, a novel signaling pathway for TxB of C. difficile in human colonic epithelial cells (FIG. 7) is described, the natural target cell of this bacterial toxin in human disease. TxB was previously shown to damage human colonocytes, but to have no effect in rat or rabbit colon. The results herein show that TxB binding to its cognate colonocyte membrane receptor activates IL-8 via EGFR-ERK signaling. Although TxA also activates ERK and IL-8, these toxins appear to bind to separate receptors and to trigger distinct signaling pathways despite their structural and enzymatic similarities.

The teachings of all the articles, patents and patent applications cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating intestinal inflammation in a mammal comprising administering to said mammal an effective amount of an agent that inhibits Clostridium difficile toxin B-mediated activation of the epidermal growth factor receptor, or an agent that inhibits Clostridium difficile toxin B-mediated activation of the extracellular signal-regulated kinase 1/2, or an agent that inhibits binding of Clostridium difficile toxin B to its cognate cell surface receptor.

2. The method of claim 1 wherein said agent is selected from the group consisting of: an epidermal growth factor receptor kinase inhibitor, an epidermal growth factor receptor antagonist, an epidermal growth factor receptor antibody or antigen-binding fragment thereof, an epidermal growth factor receptor inhibitor, an extracellular signal-regulated kinase 1/2 inhibitor and an extracellular signal-regulated kinase 1/2 antagonist.

3. The method of claim 1 wherein said agent is a matrix metalloproteinase inhibitor.

4. The method of claim 1 wherein said agent inhibits binding of TGFα to the epidermal growth factor receptor.

5. The method of claim 4 wherein said agent is selected from the group consisting of: a TGFα antagonist, a TGFα antibody or antigen-binding fragment thereof and a TGFα inhibitor.

6. A method of treating Clostridium difficile toxin B-mediated intestinal inflammation, inflammatory diarrhea, or inflammatory bowel disease in a mammal comprising administering to said mammal an effective amount of an agent that inhibits Clostridium difficile toxin B-mediated activation of the epidermal growth factor receptor, or an agent that inhibits Clostridium difficile toxin B-mediated activation of the extracellular signal-regulated kinase 1/2, or an agent that inhibits binding of Clostridium difficile toxin B to its cognate cell surface receptor.

7. The method of claim 6 wherein said agent is selected from the group consisting of: an epidermal growth factor receptor kinase inhibitor, an epidermal growth factor receptor antagonist, an epidermal growth factor receptor antibody or antigen-binding fragment thereof, an epidermal growth factor receptor inhibitor, an extracellular signal-regulated kinase 1/2 inhibitor and an extracellular signal-regulated kinase 1/2 antagonist.

8. The method of claim 6 wherein said agent is a matrix metalloproteinase inhibitor.

9. The method of claim 6 wherein said agent inhibits binding of TGFα to the epidermal growth factor receptor.

10. The method of claim 9 wherein said agent is selected from the group consisting of: a TGFα antagonist, a TGFα antibody or antigen-binding fragment thereof and a TGFα inhibitor.

11. A method of treating epidermal growth factor receptor-mediated intestinal inflammation in a mammal comprising administering to said mammal an effective amount of an agent that inhibits Clostridium difficile toxin B-mediated activation of the epidermal growth factor receptor or an agent that inhibits Clostridium difficile toxin B-mediated activation of the extracellular signal-regulated kinase 1/2, or an agent that inhibits binding of Clostridium difficile toxin B to its cognate cell surface receptor.

12. The method of claim 11 wherein said agent is selected from the group consisting of: an epidermal growth factor receptor kinase inhibitor, an epidermal growth factor receptor antagonist, an epidermal growth factor receptor antibody or antigen-binding fragment thereof, an epidermal growth factor receptor inhibitor, an extracellular signal-regulated kinase 1/2 inhibitor and an extracellular signal-regulated kinase 1/2 antagonist.

13. The method of claim 11 wherein said agent is a matrix metalloproteinase inhibitor.

14. The method of claim 11 wherein said agent inhibits binding of TGFα to the epidermal growth factor receptor.

15. The method of claim 14 wherein said agent is selected from the group consisting of: a TGFα antagonist, a TGFα antibody or antigen-binding fragment thereof and a TGFα inhibitor.
